(19) United States
(12) Patent Application Publication
(10) Pub. No.: US 2026/0290608 A1
(43) Pub. Date: Sep. 24, 2026

(54) SYSTEM AND METHOD FOR INTERPRETATION OF MEDICAL IMAGES

(71) Applicants: VOLPARA HEALTH TECHNOLOGIES LIMITED, Wellington (NZ); VOLPARA SOLUTIONS EUROPE LIMITED, Stockport (GB)

(72) Inventor: Melissa HILL, Issy les Moulineaux (FR)

(21) Appl. No.: 19/490,837

(22) PCT Filed: Jun. 6, 2024
(86) PCT No.: PCT/IB2024/055532

§ 371 Date: Dec. 5, 2025

(30) Foreign Application Priority Data
Jun. 6, 2023 (GB) 2308430.4

(51) Int. Cl. G16H 50/30; A61B 5/00; G06T 7/00; G06T 7/11; G06T 7/60

(52) U.S. Cl. CPC G16H 50/30; A61B 5/4312; A61B 5/708; G06T 7/0012; G06T 7/11; G06T 7/60; G06T 2207/30068; G06T 2207/30168

(57) ABSTRACT

A system and method for interpretation of medical images is disclosed. The method comprises reading inputs or medical images, deriving information from the medical images by analyzing individual image complexity factors using the inputs, predicting risk of missing finding using the inputs directly and the information directly, and providing outputs to users based on the risk of missing finding. The system is configured to implement the method.

### FIELD OF THE INVENTION

The present invention relates to a system and method for interpretation of medical images.

In particular it provides means to determine an image region for the evaluation of complexity according to quantitative tissue composition measurements.

It is an advantage of the present invention that clinical and technical aspects, and subject specific characteristics, help determine soft tissue imaging quality and the related image complexity that contributes to the risk of missing a finding of interest.

Further, the present invention provides explanatory data that may also be applied to adjust system parameters and image characteristics for interpretation.

### BACKGROUND

Multiple factors are known to affect the ability of clinicians to accurately interpret a medical image. For example, in mammography, these factors include the subject's breast density, the quality of breast positioning, the adequacy of breast compression, the baseline imaging system technical characteristics (noise, contrast, sharpness), the physical viewing conditions (quality of the viewing monitor, cleanliness, lighting conditions, distance of the reader from the monitor), the viewing context (time of day, distractions, what has been read previously, etc.), and the education and experience level of the reader. Each of these factors (referred to here as a ‘complexity’) can negatively impact the accuracy of image interpretation.

Elevated breast density and poor-quality patient positioning and compression have been demonstrated to be associated with a greater odds of cancers or other disease being detected between the breast cancer screening interval (referred to as interval cancers (IC)). These cancers are important to avoid, because compared to screen-detected cancers (SDC), IC are often found at a later stage when there are more limited treatment options and there is a poorer prognosis for the patient.

Good quality assurance programs attempt to mitigate any negative influence on screening performance of the factors influencing reading accuracy, but in the dynamic environment of clinical practice, errors, oversights, and uncontrollable factors may still impact interpretation such that suspicious lesions may be missed.

Recently, artificial intelligence (AI) algorithms intended to assist, or replace the human reader, in the lesion detection task have been proposed to improve breast cancer screening performance. Such automated algorithms are objective and avoid some of the image complexity factors that influence a human reader, for example, the viewing conditions, viewing context, and the variability of experience levels between readers.

However, both AI and human readers are still subject to some of the fundamental image complexity factors that influence interpretation, including breast density, the quality of breast positioning, the adequacy of breast compression, and the imaging system technical characteristics.

Breast density refers to the relative amounts of fibroglandular tissues (glandular, connective tissues) and fatty (adipose) tissues in the breast and their relative radiodensity in an image. Breast density is an individual characteristic as breast tissue composition varies between people and over the course of an individual's life.

Breast density as a parameter may be defined in several ways. Breast density may be defined in terms of absolute tissue composition. Breast density may be defined in terms of the relative tissue compositions that can be described in terms of the volumetric percentage of dense tissue, or the proportion of the imaged breast area that appears dense, according to a threshold to differentiate tissue types that can be either subjectively set, or automated as an objective parameter.

Depending on the breast positioning, including the breast compression, the breast tissues may be superimposed in an image to show areas of high radiodensity, and/or complex tissue patterns that can have the outcome of either ‘masking’ (hiding) pathological tissue, or that may mimic the appearance of a pathology.

Individuals with higher breast density are subject to their mammograms having the potential for masking. And an individual's particular parenchymal pattern, and the way this appears in an image may also contribute to the masking effect.

Multiple alternative methods exist to quantify and/or qualify breast density. As an independent fact, measures of breast density can be effective to predict masking risk and, thus, for use to select candidates for personalized screening regimens where one or more options, such as a supplemental imaging technique, or more frequent screening intervals may be proposed. Such personalized screening programs have been shown to reduce the IC rate.

Other measures of risk of developing breast cancer exist that may incorporate breast density. Widely used risk models rely on personal and familial history, along with other individual risk factors such as age, lifestyle, and breast density, and sometimes include results of genetic testing. Newer risk models use image features that may include one or more of early signs of cancer, in addition to breast density and features that describe the parenchymal pattern. The image-based risk methods can also be used in combination with the more traditional risk models to include familial and genetic information. Risk-based screening strategies have also been shown to reduce the IC rate, possibly through a combination of identifying women likely to develop more aggressive cancers and those with greater masking potential.

Methods have been developed with the specific goal of predicting masking risk that takes breast density, parenchymal pattern, imaging system characteristics, and models of visual acuity into account. The premise is that this type of multifactor model should relate more directly with the potential conspicuity of a lesion, or the ability to distinguish a lesion from the surrounding background in the image than use of either density measures or risk modelling that only have partial associations with the complexity that determines the difficulty of interpreting the mammogram. This approach is also directed towards prediction of a human reader, albeit with the upper limit of an ‘ideal’ observer that could represent machine vision.

An objective method which includes assessment of breast positioning quality or breast compression adequacy or both in the prediction would be beneficial because clinical evaluation demonstrating deficiencies in quality for each of these factors are associated with missed cancers in mammography screening. Previously, breast parenchyma has been measured on image regions selected without direct reference to tissue composition, which may limit the efficacy of those measures to adequately represent complexity in the image.

The present invention overcomes such limitations. It provides a system and method for interpretation of medical images comprising means to assess complexity of an image region according to quantitative tissue composition measurements.

### SUMMARY OF THE INVENTION

According a first aspect of the invention there is a method for interpretation of medical images of an organ assesses comprising reading inputs of medical images, deriving information from the medical images by analyzing individual image complexity factors using the inputs, predicting risk of missing finding using the inputs directly and the information directly, and providing outputs to users based on the risk of missing finding.

According to a second aspect of the invention there is a system to implement the method, comprising a data reader to read the inputs of medical images, a processor to derive the information from the medical images by analyzing individual image complexity factors using the inputs and predict the risk of missing finding using the inputs directly and the information directly, and data output display and data storage and retrieval devices to provide the outputs to users based on the risk of missing finding and save the outputs and inputs for later retrieval.

Reading the inputs may include reading clinical data for each medical image including anatomical view, patient medical data, image properties, and technical data. The system inputs may include metadata collected from the image including: information about the organ that is imaged and imaging procedures and machinery and data such as compressed breast thickness, compression force, the image acquisition technique factors, or pixel size. Reading the inputs may include reading technical system data for each medical image including system technical specifications and physical imaging system measurements.

Deriving the information may include image segmentation of features of interest for each medical image. The complexity may be assessed by applying segmentation to the image to identify the image background, an organ of interest in the image, a feature of interest in the image or in the organ, or a muscle in the image separate from the organ or a combination of these. Deriving the information may include tissue quantification in each image by estimating composition and size of the organ or feature of interest. Thus, complexity of an image or factor of the image may be assessed according to quantitative tissue composition measures. The measures may be of the organ or feature of interest. Consequently, an objective and quantitative interpretation is provided.

Tissue composition at each location of the organ or feature of interest in the image may be determined. Thus, a finding is more likely to be identified than to become a missing finding. A map of thickness of fibroglandular tissue in the organ or feature of interest may be determined. Organ or breast volume, fibroglandular tissue volume, and volumetric organ or breast density may be determined. These aids detecting and interpreting features of interest in fibroglandular tissue which otherwise be a missing finding in this tissue which may aid disease detection and prediction.

Deriving information may include image quality assessment by image sharpness evaluation, artefact evaluation, organ positioning evaluation, image noise evaluation, and image contrast evaluation. Image sharpness evaluation may be based upon immobilization of the organ evaluation. The information includes visualization evaluation of each image by tissue pattern evaluation of the organ. Image quality assessment (image level prediction) may incorporate presence of tissue with substantial pattern complexity. This may include tissue pattern assessment within image region(s) defined according to tissue composition. Consequently, there is reduced risk of missing finding in the organ or a feature of interest that would be otherwise obscured by pattern complexity of subtle variation of composition.

A proximity of the location of any image quality defect to the organ may indicate a risk of missed finding. Reducing the risk of missing finding may be aided by using tissue composition maps corresponding to multiple images obtained from a plurality of imaging modalities to determine image complexity. It may also be aided by comparing proximity of a region of image where the complexity is above a selected level to area where image quality related to selected factors is below a selected level.

Deriving information may include visualization evaluation of each image from the features of interest in each image or from the features of an examination.

Predicting risk of missing finding may include using a first model to predict missing finding of interest, a second model to predict risk of missing finding in an image ensemble, or a third model in other imaging instances and imaging modalities. The method may include applications of the risk of missed findings. The relative importance of the characteristics of image complexity factors may be used for one or more the applications of risk listed as follows: i) suggest alternative imaging technique(s) or image modality depending on the limiting characteristics; or ii) produce optimal image processing/reconstruction that minimizes image complexity; or iii) display images for interpretation according to image or exam complexity, including by ordering; or iv/assign uncertainty to quantitative measurements, both for individual metrics and those that are multifactorial; or v/classify images or image regions such that they may be prioritized for input to a model, including for new model development.

Results from the first model may be passed to the second model and results from the second model may be passed to the third model whereupon the risk of missing finding is determined.

Tissue quantification, image quality assessment, or visual evaluation all aid in determining the risk of missing finding of interest. The image quality assessment (image level prediction) may incorporate presence of an image quality defect. The image quality assessment (image level prediction) may incorporate presence of a focal density based on the tissue composition map and aid in determining whether the focal density is anomalous. Locations of the focal density may be accounted for. Therefor an anomalous focal density is less likely to be a missing finding.

A focal density may be analyzed using of an ensemble of the images to predict whether image tissue within or proximate to the focal density could be evaluated with efficacy via another modality. The image quality assessment, image level prediction, may incorporate technical data external to the image including: reading conditions, machine usage statistics, or machine physical statistics of a machine which takes or displays the image(s). The image quality assessment, image level prediction, may incorporate patient factor data external to the image including patient positioning, patient physical characteristics, apparatus that present confounding features in the image, patient prior history of interventions related to an organ of interest, or patient therapy.

A risk of missing finding may be due to or assessed by using presence of an image quality defect in more than one image of an ensemble of the medical images.

The outputs to users include the prediction of risk of missing finding. The outputs may also include a suggestion of alternative imaging technique or alternative imaging modality based on limiting characteristics, or an application of risk prediction indicators to produce optimal image processing.

The outputs to users include a suggestion of a use of the risk of missing finding. The use to be priority for interpretation. Individual features may be chosen to suggest the use to be selecting a particular one or group of the medical images or region of the organ for model development.

A displayed image features assessment with respect to tissue composition may be an output. This makes more likely a finding in a feature of interest or the organ, and the risk of missing finding in the organ or feature of interest is reduced. The risk of missing finding may be reported, saved, or output as a score. The outputs to users may include a prediction of quantitative measurement uncertainty of tissue composition, size, or disease risk.

It is an advantage of the system and method that the interpretation of medical images is improved. The medical images may include an organ of interest such as a breast, lung, or other organ comprising relatively soft tissues compared to bone.

It is a further advantage that clinical and technical factors, and subject specific characteristics help determine soft tissue imaging quality and can be used to predict the risk of missing a finding of interest.

Further, the system and method provide explanatory data that may also be applied to adjust system parameters and image characteristics for interpretation.

The method an evaluation of image visualization includes tissue pattern assessment within image region(s) defined according to tissue composition and an assessment of displayed image features, which account for image properties with respect to tissue composition.

Multiple characteristics of image complexity may be used in models to predict or explain one or more contributing factors towards a risk of missing findings of interest at an image level, or an exam level or over time, either individually or together, and either with or without the use of prior images. The method may include determining a relative importance of each of the characteristics of image complexity for predicting any of the contributing factors.

The invention will now be described, by way of example only, with reference to the accompanying figures in which:

### BRIEF DESCRIPTION OF THE FIGURES

FIG. 1 is a flow chart which illustrate the system and method for interpretation of medical images;

FIG. 2a is a mammogram of a BI-RADS 5th edition density ‘c’ breast;

FIG. 2b is a fibroglandular tissue composition map derived from the breast shown in FIG. 2a;

FIG. 2c is 407 regions of interest (ROI) relating to FIG. 2b without reference to tissue composition;

FIG. 2d is a thresholding of the tissue composition map relating to FIGS. 2b to 10% fibroglandular tissue;

FIG. 2e is 45 regions of interest (ROI) selected after thresholding located within the masked region indicated in FIG. 2d,

FIG. 2f is a thresholding of the tissue composition map relating to FIGS. 2b to 15% fibroglandular tissue; and

FIG. 2g is 24 regions of interest (ROI) selected after thresholding located within the masked region indicated in FIG. 2f.

### DETAILED DESCRIPTION OF THE INVENTION

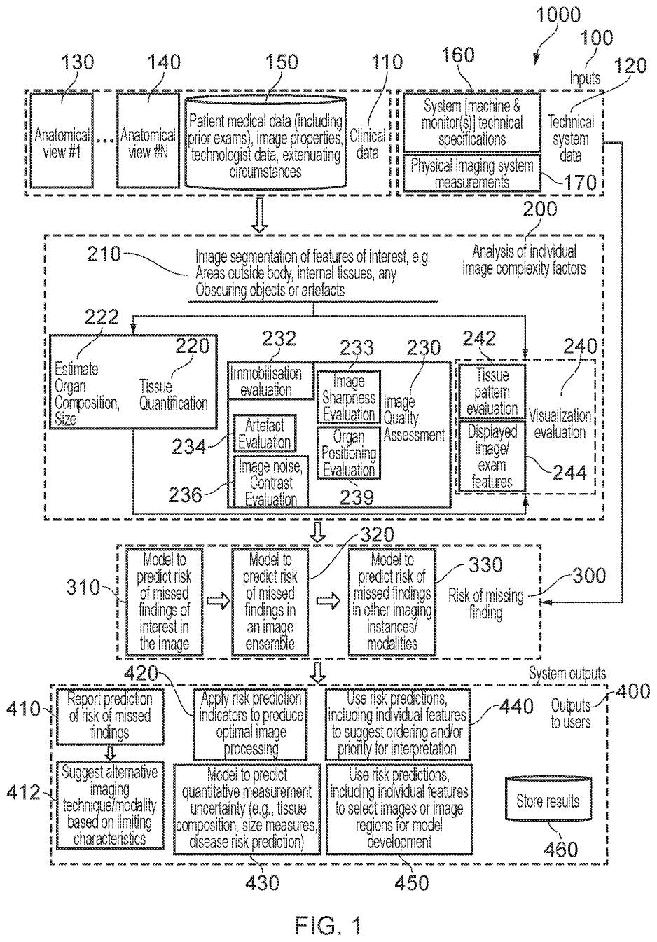

FIG. 1 illustrates by a flow chart an embodiment of a method for interpretation of medical images 1000.

The method for interpretation of medical images 1000 of an organ assesses comprising reading inputs 100 on medical images, deriving information from the medical images by analyzing individual image complexity factors 200 using the inputs 100, predicting risk of missing finding 300 using the inputs 100 directly and the information directly, and providing outputs 400 to users based on the risk of missing finding

System inputs 100 are collected including clinical data 110 and technical system data 120. The clinical data includes at least one image of a breast is acquired using radiographic techniques which includes anatomical views 130, 140, and patient medical data 150. The raw version of this image, in ‘For Processing’ format according to DICOM (Digital Imaging and Communications in Medicine) standards, is provided to the image analysis system.

Metadata 150 is collected from the image, including at least the compressed breast thickness, compression force, the image acquisition technique factors (anode/filter materials, kVp, mAs, exposure time, distances from source to patient and detector), the pixel size, numbers of image rows and columns, use of grid. The metad data includes Available technical system data 120 includes system technical specifications 160 and component physical measurements 170 are also collected.

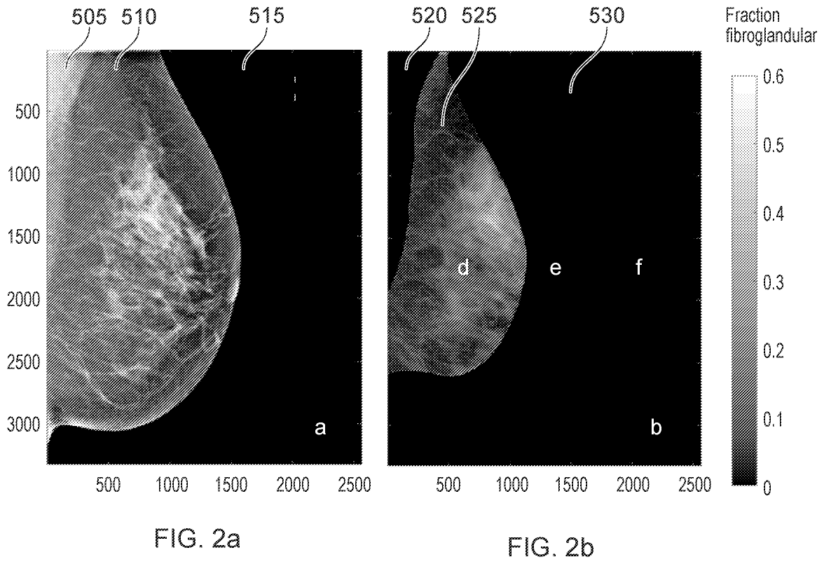

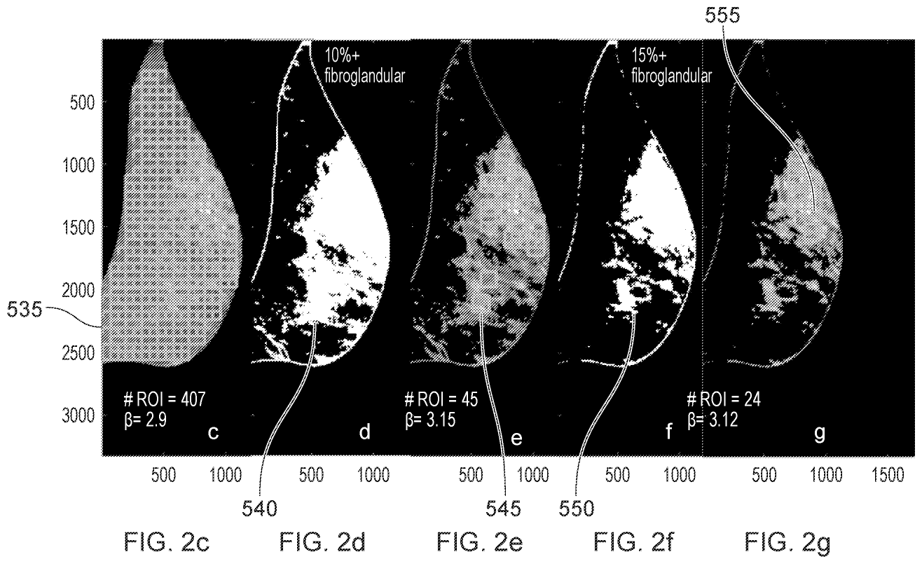

As shown in FIG. 1, image complexity factor analysis 200 uses the system inputs 100. As shown in FIG. 2, methods of image segmentation 210 are applied to identify at least, the image background 515 (may include objects distinct from the breast 510 such as the detector, compression paddle, etc.), the breast 510, and any muscle visible in the image, such as the pectoralis muscle 505. This image segmentation data will be used as an input to multiple downstream image analysis methods. For example comparing FIG. 2a to FIG. 2b, a breast 525 is segmented from the pectoralis muscle 520 and background 530.

Tissue quantification 220 is determined. An algorithm is applied to determine breast tissue composition at each image location, whereby a map of the thickness of fibroglandular tissue is generated. The image itself 130, 140, the DICOM metadata 150, and image segmentation data 210 are inputs to this algorithm. In addition to a tissue composition map, tissue quantification metrics 222 include breast volume, fibroglandular tissue volume, and volumetric breast density.

Image quality assessment 230 is determined by image quality analysis. Image quality analysis includes immobilization evaluation 232, breast positioning analysis 239, image sharpness evaluation, image noise evaluation 236, image contrast evaluation 236, artefact evaluation 233, or visualization evaluation 240 or a combination of these.

In immobilization evaluation 232, a method is applied to determine the efficacy of organ immobilisation.

As shown by the flow chart in FIG. 1, image immobilisation evaluation 232 may be conducted in parallel with artefact evaluation, image noise evaluation 236, image contrast evaluation 236, or breast positioning or organ positioning evaluation 239 or a combination of these. Image sharpness evaluation 233 may use the image immobilisation evaluation.

For example, in the case where a compression device is used for immobilisation of a breast or other body portion or organ, then the compression pressure applied during imaging can be computed. The pressure measurement may be made using a physical device (e.g. pressure sensor, or optical-based technique), or by image analysis, which may include use of one or more models to predict breast contact area with the compression paddle and compressed shape.

In either approach, the breast area in contact with the compression device surface is required, as well as either location-specific, or global measures of applied force. In the case of image-based analysis, the image metadata and image segmentation data are inputs to this algorithm. The compression pressure is then calculated as the compression force divided by the area of breast in contact with the compression paddle. Assessment of the quality of the compression pressure may be made, according to either continuous or categorical measures.

In breast positioning analysis, measures of breast positioning in the image are made. The image pixel data, the associated image metadata (e.g. DICOM header data), and image segmentation data are inputs to the positioning assessment algorithm. A series of measures of different breast positioning indicators of interest are calculated. These measures may be made according to landmarks of interest in the image to determine either quantitative measures or qualitative metrics of organ positioning. The individual measures and metrics may be combined in image-level and/or study-level positioning indicators that could be used to rank and/or classify the positioning quality.

In image sharpness evaluation 233, an evaluation of the image sharpness may be made. As described in international application PCT/IB2017/054382, the image power spectrum may be calculated for each image that is part of the exam and a comparison may be made of the relative magnitude of the noise power over a given spatial frequency range. As such, inputs to the method include image pixel data (which may include multiple image frames, or image acquisitions during a single organ immobilisation instance), associated image metadata, and image segmentation data.

Image sharpness evaluation 233 may include an assessment of any patient motion or organ motion during the image acquisition. Assessment of the organ immobilisation can be used as an input to estimate a probability of patient motion or organ motion.

Magnitude and direction of the motion may be estimated by single image analysis, or by comparison between images, and image measures, and from any immobilisation device data that may record instantaneous measures. Motion may be detected and quantified according to a flow field that assigns vectors to the motion that represent magnitude and direction, for example in accordance with the method described in international application PCT/IB2020/061379. This data may be used to interpret the likelihood of the source of the motion, such as respiratory, cardiac, and insufficient immobilisation (e.g. either patient-related or device related).

Image noise and contrast may be evaluated using approaches such as described in PCT/IB2017/054382 where the measurements are made directly from the clinical image.

Alternatively, the image pixel data, the metadata and, ideally, system physical measurements and specifications could be used to measure and/or predict the image contrast and noise using a physics-based model of the imaging system and breast. Advantageously, the tissue composition map can be used as part of the modelling to specify the tissue properties of the imaged breast.

In the artefact evaluation 234, the presence of one or more artefacts in an image may be detected by the image signal intensity, the shape of the object, or its location in the image relative to image landmarks or features. Any combination of the aforementioned properties may be used for detection.

Advantageously, detection of artefacts from highly attenuating objects (e.g. pacemaker, shoulder, metallic clips, jewelry, bones, etc.) can be made using a comparison to the expected image signal from breast tissues as determined by the image signal correspondence to the tissue composition map. Such artefacts can be excluded from the tissue composition map itself in this way using an iterative process.

Detection of artefacts with a defined shape can be made using an approach such as template matching, or through an artificial intelligence derived model that is trained to recognize the objects of interest within an appropriate level of variation of presentations of the object.

Detection of artefacts with a less defined shape and/or image signal closer to that of breast tissue can be made by comparison of the location of the potential artefact signal relative to one or more image landmarks. For example, objects located in an image region outside of the breast region, especially at the anterior side of the image, could be identified as an artefact.

Visualization evaluation 240 may include tissue pattern evaluation or examination of a displayed image or particular features of the displayed image or a combination of these.

In tissue pattern evaluation 242, visualization complexity is evaluated by means or one or more measures of organ tissue pattern complexity in the image. This metric of complexity may be determined according to image statistical features that relate to accuracy of image interpretation. One such measure is the power law parameter. This measure can be derived as the slope on a logarithmic plot of the image power spectrum versus the image spatial frequency following known methods of computation, such as described in PCT/IB2017/054382. The power spectrum computation requires an image to be analyzed in one or more regions of interest (ROI). An accepted standard is to use a series of ROI that overlap, with a windowing function applied to taper the image signal towards the ROI boundary. This approach provides multiple samples of the image of interest with good coverage.

Advantageously, the regions of interest, ROI, location(s) are selected according to the local organ tissue composition. Relevant ROI may be seen in FIG. 2. For example, in breast imaging it is well known that regions of fatty tissues appear relatively uniform in an image, whereas fibroglandular tissues most often have a more complex arrangement, with patterns that can vary between individuals and over the lifetime of an individual as can be seen in FIG. 2b. Given the greater importance of the complexity of fibroglandular tissue arrangements in the image for the efficacy of image interpretation, specific measurements of these regions may be of interest.

To incorporate tissue composition data, ROI locations for analysis are determined according to an organ tissue composition map, which is derived as part of the tissue quantification method. The tissue composition map may be segmented into areas for ROI selection. FIGS. 2c, 2e, and 2g show ROI 535, 545, 555 in within a breast.

A threshold may be applied to the tissue composition map at one or more composition ‘levels’ to capture tissue compositions of interest. FIG. 2d shows a threshold of 10%+ fibroglandular and FIG. 2f shows a threshold of 15%+ fibroglandular. In FIG. 2d an area 540 of the breast is shown that is above the 10%+ fibroglandular threshold. In FIG. 2f an area 550 of the breast is shown that is above the 15% fibroglandular threshold.

One or more image processing operations can be applied to the thresholded region, such as dilation or erosion, to create suitable region size and shape for analysis of complexity that allows for good coverage of the image region with the tissue composition of interest. For example, dilation of the thresholded tissue composition map region may be applied to permit sufficient size of connected region(s) for ROI sampling. Specific parameters to apply for region dilation may be tuned by training on representative image sets.

The power spectrum exponent is one of several potential measures of tissue pattern complexity. Other measures include a variety of image texture features and other statistical measures of the variation of image pixel intensities in the ROI. The measures may capture first, second or higher order statistical properties of the image pixels in the image area of interest. Of particular interest for a measure of complexity is the potential for correlation between image intensities at different image spatial locations. Multiple candidate metrics, and means to select appropriate metrics among multiple candidates, are well known to those skilled in the art. The measures may be defined explicitly or learned from machine learning approaches such as deep learning.

In examination of a displayed image or examination of features of a displayed image or a combination of these characteristics of the complexity of the displayed image are evaluated. The displayed image pixel data, the associated image metadata (e.g. DICOM header data), image segmentation data, and tissue quantification data are inputs. Evaluation of the characteristics may include an assessment of the size of the image area and/or volume to be interpreted relative to the breast size.

Additionally, displayed image and system resolution will be evaluated relative to the tissue composition and pattern, including local and breast-level measures.

Where reconstructed images (3D) are provided, the reconstruction properties such as the slice thickness or quality of any synthesized projection images will be evaluated relative to the local pattern complexity for the given tissue composition.

Comparisons may be made between displayed image features in an ensemble of images. The features may be evident from one or more exams. The comparison may be used to inform whether adjustment of the image processing or reconstruction parameters could be used to reduce complexity. The comparison may also be used to inform whether an alternative modality may offer reduced complexity.

Risk of missing finding 300 is determined from the analysis of individual image quality factors. The analysis of individual image quality factors produces a tissue quantification, image quality assessment, or visualization evaluation or a combination of these which are used to determine the risk of missing finding.

In determining risk of missing finding 300, there is a first model to predict risk of missing findings of interest in the image 310, a second model to predict risk of missing findings in an image ensemble 320, or a third model to predict missing findings in other imaging instances and imaging modalities 330 or a combination of these three models. Results from the first model 310 may be passed to the second model 320 and results from the second model may be passed to the third model 330 whereupon the risk of missing findings is determined.

In determining risk of missing finding there is an image level risk prediction which incorporates technical data external to the image that may affect interpretation. The technical data includes reading conditions (monitor quality, reading room lighting, etc.), machine usage statistics such as number of exposures made over the lifetime of the detector and/or x-ray tube, and machine physical measurements such as the x-ray beam quality, x-ray beam quantity, detector characteristics such as sharpness, and any defects such as dead pixels or crystallization.

The image level risk prediction incorporates patient factor data external to the image that may affect interpretation such as extenuating circumstances for imaging that limit positioning capabilities (e.g., patient in wheelchair, frozen shoulder, prominent ribs, etc.), patient physical characteristics (e.g., moles, accessory nipple, etc.) and/or any apparatus (e.g., prosthetics, pacemaker, port, etc.) that may present as confounding features in an image, a patient risk assessment for one or more clinical indications of interest, patient prior history of any interventions related to the organ of interest (e.g., biopsy, surgery, etc.), patient use of therapies (e.g., hormonal, chemotherapy, etc.),

The presence of an image quality defect (image noise, contrast, sharpness, breast positioning, artefact, and immobilisation) in the image may be used to suggest a greater risk of missed finding. The proximity of the location of any image quality defect to breast tissue in the image may also be used to suggest a greater risk of missed finding.

The presence of a focal density (continuous region of a relatively high proportion of fibroglandular tissue), based on the tissue composition map, can be used to infer greater risk of a missed finding. The proximity of the location of any image quality defect to the organ may also be used to indicate a greater risk of missed finding.

The presence of tissue with substantial pattern complexity in the image can be used to indicate a greater risk of missed finding. The presence of complexity from displayed image features can be used to indicate a greater risk of missed finding. Individual image complexity factors are combined to form the prediction of risk of missed findings, such as in a multivariate model, or by machine learned associations,

In ensemble level risk prediction, the presence of an image quality defect in more than one image of the same organ may be used to indicate a greater risk of missing finding the image quality defect.

In an ensemble level risk prediction, the presence of a focal density (continuous region of a relatively high proportion of fibroglandular tissue), based on the tissue composition map, in more than one image of the same organ such as breast, lung, or another soft tissue organ, may be used to infer greater risk of a missed finding, especially with correspondence between the locations of the density according to the image acquisition geometries of each image under evaluation.

In an ensemble level risk prediction, the complexity factors from each image in the image ensemble, may be combined to form the prediction of risk of missed findings, such as in a multivariate model, or by machine learned associations.

Missed findings in other imaging instances and modalities may be predicted. The tissue composition may be used to predict the potential image complexity by using additional images or using other image modalities or a combination additional images and other image modalities. The image ensemble evaluation of focal density can be used to predict whether imaged tissue within or proximate to the focal density could be evaluated with efficacy on a different modality. The patient extenuating circumstances would be used to predict the image complexity in additional views using either the same modality, or that an alternative imaging modality could produce.

In a model that uses tissue composition in combination with patient age, body mass index, and potentially other risk factors, the risk of missed findings in future imaging instances with the same image modality can be made.

Models to account for the likely tissue visualization in other modalities such as ultrasound, magnetic resonance imaging, etc. with reference to tissue composition, breast positioning, patient compliance, displayed image properties, may be incorporated in the prediction of missed findings.

Outputs 400 may be reported to user

A risk of missing finding may be reported 410 to the user as a score. A score may be available for individual images that are part of any ensemble under analysis. The score may represent a collective evaluation of any ensembled of images under analysis. A score may be accompanied by a description of the relative importance of any and all individual, or aggregate, characteristics that contribute to the risk estimate.

Decision support may be offered based on an evaluation of the relative importance of characteristics that contribute to the risk estimate, such as alternative imaging technique(s) 412 that could achieve a lower risk, or a suggestion of an alternative imaging modality upon an evaluation that the contributing characteristics cannot be mitigated by technique adjustment otherwise.

In particular, landmarks determined as part of the image quality assessment may be used to compare proximity of a region of image ‘complexity’ to areas with deficiencies in image quality related to factors including but not limited to, the presence of artefacts, blur, skin folds, high noise, low contrast, low compression, and tissue cutoff. The proximity may indicate the potential for greater uncertainty in image interpretation, and this uncertainty may necessitate retake of the image. A retake decision may be informed by the particular image quality deficiency that is of greatest importance in the interpretation uncertainty estimate.

The risk prediction or individual metrics derived in this method or a combination of them may be used. They may inform image processing for optimal output or display 420 to mitigate the risk of limited potential for visualization, where possible. Preferably the image processing characteristics are specifically informed by the importance of the characteristics to the risk estimate.

Specifically, a determination may be made of the sufficiency of image display format for the tissue composition and complexity at each image location. For example, in projection imaging (2D), the image contrast, locally or globally in the image, may be adjusted to adapt for the given image complexity.

Adjustments to the image reconstruction parameters may be determined from this process, such as optimal slice thicknesses and reconstruction filters. Visualization evaluation may include tissue pattern evaluation or examination of a displayed image or examination of features of a displayed image. Visualization evaluation may indicate that image reconstruction parameters will not reduce complexity. Otherwise, visualization examination may indicate that alternative imaging techniques including acquisition parameters or geometry or position of the patient or organ are preferred.

Risk predictions of individual features 440 may be used to suggest ordering or priority for interpretation. For example, images or exams with specific complexity features may be grouped for superior visual adaptation (i.e., where a human reader becomes accustomed to a particular image signal level, or level of complexity in the image, such as from the tissue pattern), for interpretation at a certain time, for reading by clinicians with particular profiles, or by algorithms with advantageous performance on those characteristics.

Risk predictions of individual features or a combination of these 450 may be used to select images, image regions, or an ensemble of images or regions for use in model or algorithm development that may include artificial intelligence models such as for feature detection, classification, or prediction of disease.

The invention has been described by way of examples only. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the claims.

1. A method for interpretation of medical images of an organ comprising reading inputs of medical images, deriving information from the medical images by analyzing individual image complexity factors using the inputs, predicting risk of missing finding using the inputs directly and the information directly, and providing outputs to users based on the risk of missing finding.

2. A method according to claim 1 wherein reading the inputs includes reading clinical data for each medical image including anatomical view, patient medical data, image properties, and technical data.

3. A method according to claim 2 wherein reading the inputs includes reading technical system data for each medical image including system technical specifications and physical imaging system measurements.

4. A method according to claim 3 wherein deriving information includes image segmentation of features of interest for each medical image.

5. A method according to claim 4 wherein deriving the information includes tissue quantification in each image by estimating composition and size of the organ or the features of interest.

6. A method according to claim 5 wherein deriving the information includes image quality assessment by image sharpness evaluation, artefact evaluation, organ positioning evaluation, image noise evaluation, and image contrast evaluation.

7. A method according to claim 6 wherein image sharpness evaluation is based upon immobilization of the organ evaluation.

8. A method according to claim 7 wherein deriving the information includes visualization evaluation of each image by tissue pattern evaluation of the organ.

9. A method according to claim 7 wherein deriving the information includes visualization evaluation of each image from the features of interest in each image or from features of an examination.

10. A method according to claim 5 wherein predicting risk of missing finding includes using a first model to predict missing finding of interest, a second model to predict risk of missing finding in an image ensemble, or a third model in other imaging instances and imaging modalities.

11. A method according to claim 10 wherein results from the first model are passed to the second model and results from the second model are passed to the third model whereupon the risk of missing finding is determined.

12. A method according to claim 5 wherein the outputs to users include the prediction of risk of missing findings.

13. A method according to claim 12 wherein the outputs include a suggestion of alternative imaging technique or alternative imaging modality based on limiting characteristics.

14. A method according to claim 5 wherein the outputs to users include an application of risk prediction indicators to produce optimal image processing.

15. A method according to claim 5 wherein the outputs to users include a prediction of quantitative measurement uncertainty of tissue composition, size, or disease risk.

16. A method according to claim 5 wherein the outputs to users include a suggestion of a use of the risk of missing finding.

17. A method according to claim 16 including choosing individual features of interest to suggest use to be priority for interpretation.

18. A method according to claim 16 including choosing individual features of interest to suggest the use to be selecting a particular one or group of the medical images or region of the organ for model development.

19. A system to implement a method for interpretation of medical images of an organ, the system comprising a data reader to read the inputs of medical images, a processor to derive the information from the medical images by analyzing individual image complexity factors using the inputs and predict the risk of missing finding using the inputs directly and the information directly, and data output display and data storage and retrieval devices to provide the outputs to users based on the risk of missing finding and save the outputs and inputs for later retrieval.
